(19) United States
(12) Patent Application Publication
(10) Pub. No.: US 2023/0218871 A1
(43) Pub. Date: Jul. 13, 2023

(54) DRAINAGE SHUNT DEVICES AND METHODS FOR DRAINING ASCITES FLUID FROM PERITONEAL CAVITIES

(71) Applicant: CareFusion 2200, Inc., San Diego, CA (US)

(72) Inventors: Gianluca Puliti, Mundelein, IL (US); Hong Shen, Hoffman Estates, IL (US); Kelly Hurtis, Chicago, IL (US); Briana Flanagan, Niles, IL (US)

(73) Assignee: CareFusion 2200, Inc., San Diego, CA (US)

(21) Appl. No.: 17/996,748

(22) PCT Filed: Apr. 20, 2021
(86) PCT No.: PCT/US2021/028097

§ 371 Date: Oct. 20, 2022

Related U.S. Application Data
(60) Provisional application No. 63/014,204, filed on Apr. 23, 2020.

(51) Int. Cl. A61M 27/00; A61M 25/00

(52) U.S. Cl. CPC A61M 27/002; A61M 25/0082; A61M 2210/1017

(57) ABSTRACT

A shunt for draining fluid including a first catheter, an electronic pump fluidly coupled to the first catheter, a manual pump fluidly coupled to the electronic pump, a second catheter fluidly coupled to the manual pump. During a drainage operation, the shunt arranged and configured such that a fluid is passively pressure-driven through the shunt, and the electronic pump is arranged to prevent blockages within the shunt by flowing a bolus of the fluid through the shunt. Additionally, the manual pump is arranged to prevent blockages within the shunt by flowing a bolus of the fluid through the shunt in the event that the electronic pump cannot produce a bolus of the fluid.

### CROSS REFERENCE TO RELATED APPLICATIONS

This application claims priority to U.S. Provisional Patent Application No. 63/014,204, filed on Apr. 23, 2020, the entire disclosure of which is hereby incorporated by reference.

### TECHNICAL FIELD

The present specification generally relates to drainage shunt devices and methods for draining ascites fluid from peritoneal cavities and, more specifically, to peritoneal shunt devices to drain non-malignant ascites fluids and methods for draining non-malignant ascites fluids.

### BACKGROUND

Shunt devices are used to drain fluid from an area within the body. Specifically, peritoneal shunt devices are used to drain ascites fluid from the peritoneal cavity of a subject. Ascites is the accumulation of fluid within the abdomen. In order to drain the fluid from the peritoneal cavity, shunt devices may rely on electronic pumps which provide consistent pressure to pump the fluid from the peritoneal cavity to the central venous system or the bladder of the subject. However, since the pump would always be activated in order to drain the fluid, the battery life of the pump diminishes, and needs recharging. If the pump were to stop functioning, no fluid would be drained from the peritoneal cavity, causing complications to the subject. Even further, blockages may form within the shunt devices as fluid passes through them. In order to clear the blockages within the shunt, varying pressures must be produced within the shunt.

Accordingly, there is a need for peritoneal shunt devices that drain fluid from the peritoneal cavity and clear blockages within the shunt passively.

### SUMMARY

According to a first aspect, a shunt for draining fluid, including a first catheter, an electronic pump fluidly coupled to the first catheter, a manual pump fluidly coupled to the electronic pump, and a second catheter fluidly coupled to the manual pump. During drainage operation, a fluid is passively pressure-driven through the shunt, and the electronic pump is arranged to prevent blockages within the shunt by flowing a bolus of the fluid through the shunt.

According to a second aspect, a shunt according to the previous aspect, further comprising a control unit that operates the electronic pump at discrete time intervals.

According to a third aspect, a shunt according to any of the previous aspects, wherein the electronic pump moves the bolus of fluid as a pressure wave of the fluid flowing through the shunt at a greater pressure than the fluid being pressure-driven through the shunt.

According to a fourth aspect, a shunt according to any of the previous aspects, further comprising a one-way valve fluidly coupled to the second catheter.

According to a fifth aspect, a shunt according to any of the previous aspects, wherein the first catheter, the electronic pump, the manual pump, the second catheter, and the one-way valve are serially arranged to form the shunt.

According to a sixth aspect, a shunt according to any of the previous aspects, wherein the first catheter is configured to be arranged within a peritoneal space of a subject in order to drain fluid from the peritoneal space.

According to a seventh aspect, a shunt according to any of the previous aspects, wherein the second catheter is configured to be arranged within a bladder of the subject in order to fluidly couple the peritoneal space of the subject with the bladder of the subject.

According to an eighth aspect, a shunt according to any of the previous aspects, wherein the one-way valve inhibits fluid within the bladder from flowing through the shunt.

According to a ninth aspect, wherein the manual pump is arranged and configured to move a bolus of the fluid through the shunt based on a manual activation.

According to a tenth aspect, a shunt for draining a fluid from a peritoneal cavity, including a first catheter, an electronic pump, a manual pump, a second catheter, and a one-way valve. The first catheter is configured to be arranged within the peritoneal cavity of a subject. The electronic pump is fluidly coupled to the first catheter. The manual pump is fluidly coupled to the first catheter. The second catheter is configured to be arranged within a bladder of a subject and is fluidly coupled to the electronic pump and the manual pump. A one-way valve is fluidly coupled to the second catheter and configured to inhibit a back-flow of the fluid from the bladder through the second catheter. The electronic pump is arranged and configured to move a bolus of the fluid in order to inhibit blockages within the shunt. The manual pump is arranged and configured to move a bolus of the fluid in order to inhibit blockages within the shunt when the electronic pump cannot create the bolus of fluid.

According to an eleventh aspect, a shunt according to any of the previous aspects, further comprising a control unit that operates the electronic pump at preselected time intervals.

According to a twelfth aspect, a shunt according to any of the previous aspects, wherein the electronic pump moves the bolus of fluid as a pressure wave of the fluid flowing through the shunt at a greater pressure than the fluid being passively pressure-driven through the shunt.

According to a thirteenth aspect, a shunt according to any of the previous aspects, wherein the electronic pump and the manual pump are arranged and configured to be outside of the peritoneal cavity.

According to a fourteenth aspect, a shunt according to any of the previous aspects, wherein the first catheter, the electronic pump, the manual pump, the second catheter, and the one-way valve are serially arranged to form the shunt.

According to a fifteenth aspect, a shunt according to any of the previous aspects, wherein the electronic pump and the manual pump are fluidly coupled in parallel.

According to a sixteenth aspect, a method of draining a peritoneal cavity of a fluid includes arranging a first catheter within the peritoneal cavity of a subject and arranging a second catheter within a bladder of the subject. The first catheter and the second catheter are fluidly coupled. The fluid from the peritoneal cavity is passively moved to the bladder through the first catheter and the second catheter due to a pressure differential between the peritoneal cavity and the bladder. A first bolus of fluid is moved via an electronic pump fluidly coupled to first catheter and the second catheter. A second bolus of fluid is moved via a manual pump fluidly coupled to the first catheter and the second catheter when the electronic pump cannot create the first bolus of fluid. The first bolus of fluid or the second bolus of fluid is flowed through the second catheter in order to reduce a blockage within the second. catheter

According to a seventeenth aspect, a method of draining a peritoneal cavity of a fluid according to any of the previous aspects, wherein the first bolus of fluid and the second bolus of fluid are moved at preselected time intervals.

According to an eighteenth aspect, a method of draining a peritoneal cavity of a fluid according to any of the previous aspects, wherein the first bolus of fluid and the second bolus of fluid are pressure waves of the fluid flowing through the second catheter at a greater pressure than the fluid flowing from the peritoneal cavity to the bladder through the second catheter due to the pressure differential between the peritoneal cavity and the bladder.

According to a nineteenth aspect, a method of draining a peritoneal cavity of a fluid according to any of the previous aspects, wherein a one-way valve is fluidly coupled to the second catheter in order to inhibit a back-flow of the fluid from the bladder through the second catheter.

According to a twentieth aspect, a method of draining a peritoneal cavity of a fluid according to any of the previous aspects, wherein the electronic pump and the manual pump are only used to move the first bolus of the fluid or the second bolus of the fluid.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage shunt device, according to one or more embodiments shown or described herein;

FIG. 2 schematically depicts the drainage shunt device of FIG. 1 arranged within a subject, according to one or more embodiments shown or described herein;

FIG. 3A schematically depicts a drainage shunt device, according to one or more embodiments shown or described herein;

FIG. 3B schematically depicts a drainage shunt device, according to one or more embodiments shown or described herein;

FIG. 3C schematically depicts a drainage shunt device, according to one or more embodiments shown or described herein; and

FIG. 4 depicts a method of using the drainage shunt device of FIG. 1, according to one or more embodiments shown or described herein.

### DETAILED DESCRIPTION

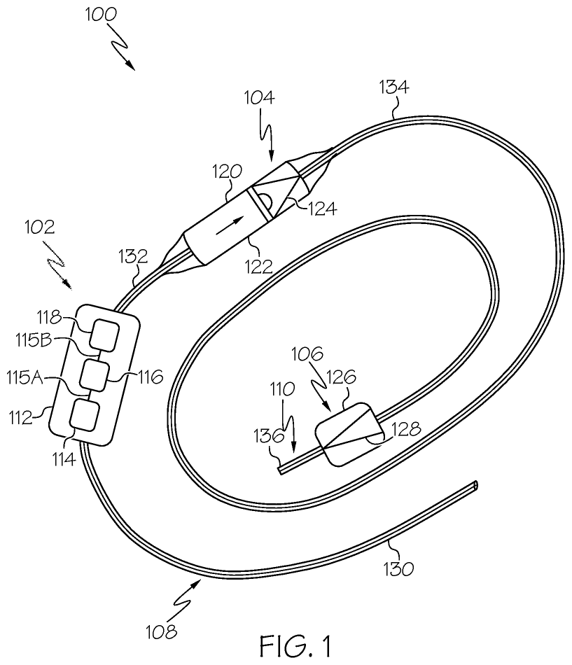

FIG. 1 generally depicts an embodiment of a shunt device for draining non-malignant ascites fluid. The shunt device generally includes an electronic pump, a manual pump, a one-way valve, a first catheter, and a second catheter. As will be described in greater detail herein, the shunt device may be used in order to drain ascites fluid from the peritoneal cavity of a subject passively to the bladder of a subject, in order to remove the ascites fluid from the subject's body. For example, the first catheter may be arranged within the peritoneal cavity, and the second catheter may be arranged within the bladder, fluidly coupling the peritoneal cavity to the bladder such that ascites fluid from the peritoneal cavity flows to the bladder due to a pressure gradient between the peritoneal cavity and the bladder and positioning of the shunt device. The term “passively” as used to describe the flow of fluid through the shunt device is defined as moving the fluid through the shunt device without the need for external pumps. The passive pumping action occurs due, at least in part, to internal forces generated by the subject's body, such as by breathing, exerting their diaphragm, a natural pressure differential between two separate cavities, and/or surface tension within the shunt device. Furthermore, the electronic pump and manual pump may be arranged to create a bolus of fluid at varying time intervals in order to clear any blockages that may have formed within the shunt device. Various embodiments of the shunt device and methods will be described in greater detail herein.

Referring now to FIG. 1, an embodiment of a shunt device 100 is generally depicted. As illustrated, the shunt device 100 may include an electronic pump 102, a manual pump 104, a one-way valve 106, a first catheter 108, and a second catheter 110. As will be described in greater detail herein, the shunt device 100 is configured to passively drain ascites fluid from the peritoneal cavity of a subject to the bladder of the subject in order to remove the ascites fluid from the body and inhibit blockages from forming within the shunt device 100. It is noted that the present shunt device may be used to drain any type of fluid from and to any type of cavity/area within a subject's body. It is noted that the shunt device 100 may drain fluid to the bladder. However, it is contemplated that the present shunt device 100 may be used to drain fluid to other areas of the body, such as the central venous system.

Referring still to FIG. 1, the electronic pump 102 may include a housing 112, a motor 114, a control unit 116, and a battery 118. The motor 114 is communicatively coupled to the control unit 116 and the battery 118 via wires 115A, 115B. The motor 114 is fluidly coupled to the first catheter 108 such that when the motor 114 is activated by the control unit 116, a bolus of fluid formed from the fluid within the first catheter 108 is created. The control unit 116 may be a pre-programmed computer which is designed to activate the motor 114 at specific and discrete time intervals. For example, the control unit 116 may be programmed to activate the motor 114 in 12-hour increments for a duration of one minute. Additionally, the control unit 116 may be reprogrammed after surgical insertion into a subject via a wireless connection, such as Wi-Fi or Bluetooth, if the operating parameters of the electronic pump 102 need to be altered. The battery 118 of the electronic pump 102 is operatively arranged to provide power to both the motor 114 and the control unit 116. The battery 118 may be a lithium ion battery type, which may be wirelessly rechargeable through induction charging. The motor 114, control unit 116, and battery 118 are arranged within the housing 112. The housing 112 may be a rigid or flexible shell, which seals the motor 114, control unit 116, and battery 118 form the outside environment, such as inside of a subject's body. The housing 112 may be made form a non-conductive material to allow for wireless communication with the control unit 116 and induction charging of the battery 118.

The electronic pump 102 is fluidly coupled to the first catheter 108 to draw fluid to flow from the first catheter 108 through the electronic pump 102. The first catheter 108 includes a tip portion 130, which is arranged to be placed within the peritoneal cavity of a subject. In embodiments, the first catheter 108 is long enough to arrange the tip portion 130 within the peritoneal cavity of a subject, while allowing the electronic pump 102 to be arranged outside of the peritoneal cavity, but within a subject's body. The first catheter 108 may be any type of flexible tubing suitable to be inserted within a subject's body. Additionally, in embodiments, the first catheter 108 may be shaped in various forms in order to secure the first catheter 108 within a peritoneal cavity. For example, the tip portion 130 may be bent in a curve or spiral shape in order to secure the tip portion 130 within a peritoneal cavity.

Referring still to FIG. 1, the manual pump 104 may include a housing 120, a flexible membrane 122, and a valve 106. The flexible membrane 122 is used to manually pump fluid through the shunt device 100 by depressing the flexible membrane 122. By depressing the flexible membrane 122, a pressure is created within the housing 120, which causes the fluid contained within the shunt device 100 to flow towards the bladder of a subject. The fluid flows from the housing 120 through the valve 106. The valve 106 prevents back-flow of any fluid passing through the manual pump 104 from flowing back towards the peritoneal cavity. The valve 106 may be a duckbill valve, or another type of one-way valve which only allows fluid to flow in a single direction when a pressure differential is created on opposite sides of the valve 106. In embodiments, the manual pump 104 may be arranged in line with the electronic pump 102, where fluid from the peritoneal cavity would pass through the electronic pump 102 prior to the same fluid entering the manual pump 104. The manual pump 104 is fluidly coupled to the electronic pump 102 via tubing 132. Tubing 132 may be any flexible tubing which is suitable for insertion into a subject's body. In some embodiments, the manual pump 104 may be directly fluidly coupled to the electronic pump 102, without the need for tubing 132.

Referring still to FIG. 1, another one-way valve 124 is arranged to be fluidly coupled to the electronic pump 102 and the first catheter 108. The fluid flows from the housing 112 through the valve 124. The valve 124 prevents back-flow of any fluid passing through the electronic pump 102 from flowing back towards the peritoneal cavity. The valve 124 may be a duckbill valve, or another type of one-way valve which only allows fluid to flow in a single direction when a pressure differential is created on opposite sides of the valve 124.

Arranged on the opposite side of the one-way valve 106 from the manual pump 110 is the second catheter 110. The second catheter 110 includes a tip portion 136 which is inserted into the bladder of a subject. The second catheter 110 may be any type of flexible tubing suitable to be inserted within a subject's body. Additionally, in embodiments, the second catheter 110 may be shaped in various forms in order to secure the second catheter 110 within a peritoneal cavity. For example, the tip portion 136 may be bent in a curve or spiral shape in order to secure the tip portion 136 within a peritoneal cavity.

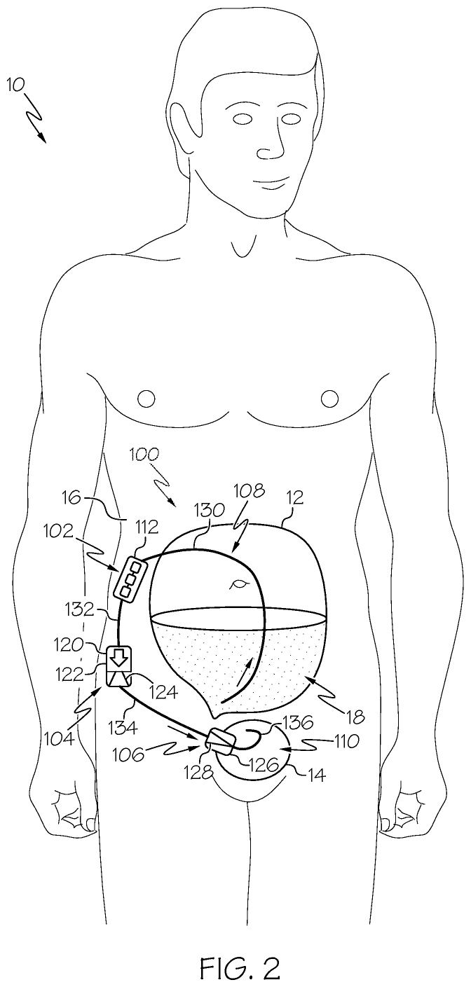

Referring now to FIG. 2, the shunt device 100 may be arranged within the body 16 of a subject 10 in order to drain ascites fluid 18 from the peritoneal cavity 12 to the bladder 14. Specifically, the first catheter 108 may be arranged within the peritoneal cavity 12 of the subject 10. The peritoneal cavity 12 is a space between the parietal peritoneum (the peritoneum that surrounds the abdominal wall) and the visceral peritoneum (the peritoneum that surrounds the internal organs) within the body 16 of the subject 10. However, in embodiments, the first catheter 108 may be arranged in any area of the body 16 which requires a fluid to be drained. The first catheter 108 is arranged within the peritoneal cavity 12 is such a way that the fluid 18 can enter the first catheter 108. For example, the tip portion 130 of the first catheter 108 may be arranged in the lower section of the peritoneal cavity 12 where the fluid 18 collects within the peritoneal cavity 12. The first catheter may also extend through an incision within the wall of the peritoneal cavity 12. The electronic pump 102 may be arranged outside of the peritoneal cavity 12, and more specifically, may be arranged just underneath the skin of the body 16 to allow for wireless charging or communication with the electronic pump 102. Additionally, the manual pump 104 may be arranged just underneath the skin of the body 16 in order to allow the subject 10 to depress the flexible membrane 122 of the manual pump 104 in order to flow a bolus of fluid through the shunt to prevent blockages.

Referring still to FIG. 2, the second catheter 110 may be arranged within the bladder 14 of the subject 10. As fluid 18 drains from the peritoneal cavity 12 to the bladder 14 through the shunt device 100, the bladder will begin to fill with the fluid 18. As the bladder 14 fills to capacity, and prior to the subject 10 emptying their bladder 14, there is the possibility that the fluid 18 stored within the bladder 14 will reach the tip portion 136 of the second catheter 110 and attempt to flow in a reverse direction through the shunt device 100. The one-way valves 106 and 124 of each pump 102 and 104 prevent the back-flow of fluid 18 from the bladder through the shunt device 100.

Due to the arrangement of the shunt device 100 within the body 16 of the subject 10, with the first catheter 108 arranged within the peritoneal cavity 12 and the second catheter 110 arranged within the bladder 14, a passive pressure-driven flow of fluid 18 can be formed between the peritoneal cavity 12 and the bladder 14 through the shunt device 100. Since the peritoneal cavity 12 and the bladder 14 are fluidly coupled, any pressure gradient between the peritoneal cavity 12 and the bladder 14 will create a pressure-driven flow of fluid 18. For example, as pressure increases within the peritoneal cavity 12, the fluid 18 stored within the peritoneal cavity 12 will be forced through the first catheter 108 as a pressure release for the peritoneal cavity 12. The pressure within the peritoneal cavity 12 may be increased by having the subject exert their diaphragm into their abdominal cavity, or by breathing normally. Since the action of breathing or exerting a diaphragm does not increase the pressure within the bladder 14, a pressure gradient is formed between the peritoneal cavity 12 and the bladder 14, with the high pressure being present in the peritoneal cavity 12, and the low pressure being present in the bladder 14. This difference in pressures passively flows the fluid 18 from the peritoneal cavity 12 to the bladder 14, without the need of additional pumping mechanisms. Due to the arrangement of the electronic pump 102 and the manual pump 104 being in fluid communication, the passive pressure driven flow may still flow through the shunt device 100 even when the pumps are not activated.

As the fluid 18 flows from the peritoneal cavity 12 to the bladder 14 through the shunt device 100, biological material may be present within the fluid and can form potential blockages within the shunt device 100 as the biological material collects on the inner surfaces of the fluid channels forming the shunt device 100. In order to prevent blockages, or to clear blockages, a bolus of fluid may be created and flowed through the components of the shunt device 100 in order to remove the biological matter to the bladder 14. A bolus of fluid is a pressure wave of the fluid 18 flowing through the shunt device 100 at a greater pressure than the fluid 18 being passively pressure-driven through the shunt device 100. The bolus of fluid may be formed by either the electronic pump 102 and/or the manual pump 104. In embodiments, the electronic pump 102 is programed to activate and create a bolus of fluid at discrete time intervals throughout the day. The purpose of having discrete time intervals to create a blockage clearing bolus is to prevent blockages from forming initially. Since the electronic pump 102 only activates for short periods of time, and only a few times per day, the battery life of the electronic pump 102 may be extended when compared to shunt devices which routinely require a pump to drain the fluid 18 from the peritoneal cavity 12 at a constant rate.

In the event that the electronic pump 102 cannot create a bolus of fluid to prevent or clear blockages within the shunt device 100, the manual pump 104 may be used to create the bolus of fluid. Situations where the electronic pump 102 may fail include a malfunction with the electrical components, the mechanical components, or a depleted battery. In these situations, without the presence of the manual pump 104, the subject 10 would be required to undergo immediate surgery to replace the electronic pump 102, otherwise excessive fluid 18 may build up within the peritoneal cavity 12 of the subject, which may cause other related health problems. The manual pump 104 may be used to create a bolus of fluid, of the same or greater pressure than that of the electronic pump 102, in order to prevent or clear blockages within the shunt device 100. If the manual pump 104 is required to be used, the subject 10 should pump the manual pump 104 at discrete time intervals throughout the day of a set number of pumps. For example, the subject should pump the manual pump 104 every twelve hours for at least twenty individual pumps. This would create a sufficient bolus of fluid 18 to clear any biological material within the shunt device 100.

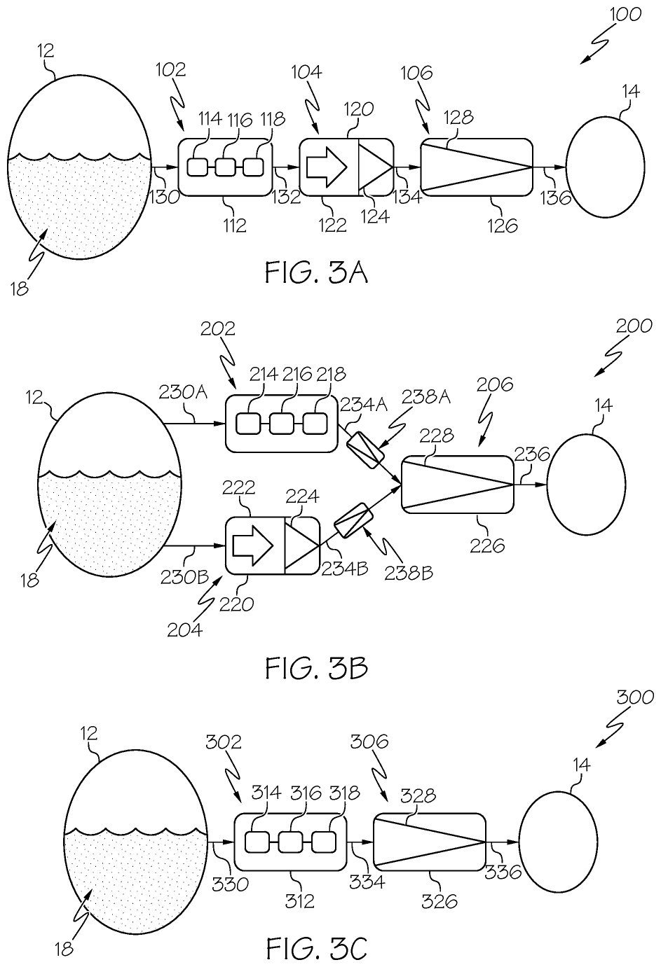

Referring now to FIGS. 3A-3C, various components forming a shunt device can be arranged to achieve different results, depending on the required parameters of the subject 10. As depicted in FIG. 3A, the electronic pump 102, manual pump 104, and one-way valve 106 are in a series arrangement, where fluid 18 from the peritoneal cavity 12 passes through each component of the shunt device 100. The fluid 18 will still pass through the electronic pump 102 and the manual pump 104 even when the pumps are not creating a bolus of fluid.

Referring now to FIG. 3B, a shunt device 200 may include an electronic pump 202 and a manual pump 204 in parallel arrangement to the peritoneal cavity 12. The electronic pump 202 is fluidly coupled to the peritoneal cavity 12 via catheter 230A. The manual pump 204 is fluidly coupled to the peritoneal cavity 12 via the catheter 230B. The electronic pump 202 is fluidly coupled to a one-way valve 224 to prevent back-flow. The manual pump 204 is fluidly coupled to a one-way valve 206 to prevent back-flow.

Referring now to FIG. 3C, a shunt device 300 may include an electronic pump 302 that includes a motor 314, a control unit 316, and a battery 318 communicatively coupled to one another in order to activate the electronic pump 302. The electronic pump 302 is fluidly coupled to the peritoneal cavity via catheter 330. The one-way valve 324 is serially arranged with the electronic pump 302 and is fluidly coupled to the electronic pump 302. The one-way valve 324 includes a housing 326 and valve members 328 to prevent back-flow of fluid 18 from the bladder 14, and is fluidly coupled to the bladder 14 via catheter 336.

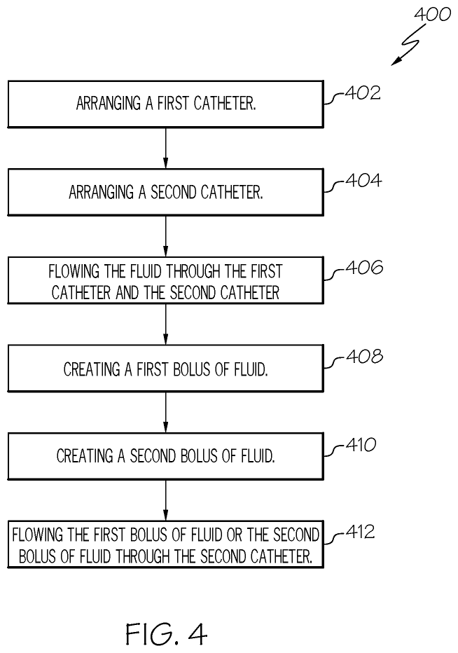

Referring now to the flow diagram of FIG. 4 in conjunction with FIGS. 1 and 2, an illustrative method 400 of draining ascites fluid from a peritoneal cavity to a bladder of a subject is schematically depicted. More specifically, a shunt device 100 is operable to passively drain fluid 18 from a peritoneal cavity 12, while also preventing blockages from forming within the shunt device 100. The depiction of FIG. 4 and the accompanying description below is not meant to limit the subject matter described herein or represent an exact description of how the shunt device 100 operates, but instead is meant to provide a simple schematic overview to illustrate the general passive drainage of fluid and blockage prevention measures of the method described herein.

Referring still to FIG. 4, in conjunction with the shunt device 100 of FIGS. 1 and 2, a flow diagram is schematically depicted of an illustrative method 400 of draining a peritoneal cavity 12 of a fluid 18. Initially, at step 402, a first catheter is arranged with a peritoneal cavity of a subject. Referring to FIG. 2, the first catheter 108 includes a tip portion 130 which may be arranged within the peritoneal cavity 12 of a subject 10. The first catheter 108 fluidly couples the remaining components of the shunt device 100 to the peritoneal cavity 12.

At step 404, a second catheter is arranged within a bladder of the subject. Referring to FIG. 2, the second catheter 110 include a tip portion 136 and is arranged within the bladder 14 of the subject 10. The first catheter 108 and the second catheter 110 are fluidly coupled to one another via the components which create the shunt device 100.

At step 406, fluid is flowed form the peritoneal cavity to the bladder. Referring to FIG. 2, the first catheter 108 and the second catheter 110 fluidly couple the peritoneal cavity 12 to the bladder 14. The fluid 18, which is present in the peritoneal cavity 12, flows through the first catheter 108 and the second catheter 110 due to a pressure differential between the peritoneal cavity 12 and the bladder 14. The pressure differential is created by the subject 10 increasing pressure within their abdominal cavity by either breathing or exerting their diaphragm into the abdominal cavity. The fluid 18 flows continuously through the first catheter 108 and the second catheter 110 in order to continuously drain the peritoneal cavity 12.

Still referring to FIG. 4, at step 408, a first bolus of fluid is created. Referring to the example being described in the preceding paragraphs, a bolus of fluid 18 may be created at discrete time intervals by the electronic pump 102 atomically in order to prevent and clear any biological material which collects within the shunt device 100. The bolus of fluid is a pressure wave of fluid 18 travelling through the second catheter 110 at a greater pressure than the passively drained fluid 18 flowing through the shunt device 100 due to the pressure differential between the peritoneal cavity 12 and the bladder 14. The greater pressure of the bolus of fluid 18 dislodges or removes any biological material present within the tubing of the shunt device 100, and removes the biological material to the bladder 14 to be expelled from the subject 10.

Still referring to FIG. 4, at step 410, a second bolus of fluid is created. Referring to the example being described in the preceding paragraphs, a second bolus of fluid 18 may be created by the manual pump 104. In embodiments, the manual pump 104 is only used in the event that the electronic pump 102 malfunctions or loses power, and is unable to produce a bolus of fluid 18. If the manual pump 104 is used to create a bolus of fluid 18, the flexible membrane 122 of the housing 120 is depressed in order to create a pressure differential between the fluid 18 within the housing 120 and the fluid within the second catheter 110. Since the pressure is greater within the housing 120, the bolus of fluid 18 will be created in the housing 120 and be biased to travel through the shunt device 100 towards the second catheter 110.

At step 412, the first bolus of fluid or the second bolus of fluid are flowed through the second catheter. Referring to the example described in the preceding paragraphs, a bolus of fluid 18 is propagated through the tubing of the shunt device 100 in order to remove any biological material which may form a blockage within the shunt device 100. Depending on the bolus creating state of the electronic pump 102, the bolus of fluid 18 may be create using the manual pump 104. If the electronic pump 102 is capable of creating a bolus of fluid 18, then the manual pump 104 would not be required to create a bolus of fluid 18.

It should now be understood that embodiments described herein are directed to a shunt device for draining non-malignant ascites fluid. The shunt device includes an electronic pump, a manual pump, a one-way valve, a first catheter, and a second catheter. The shunt device may be used in order to drain ascites fluid from the peritoneal cavity of a subject to the bladder of a subject, in order to remove the ascites fluid from the subject's body. For example, the first catheter may be arranged within the peritoneal cavity, and the second catheter may be arranged within the bladder, fluidly coupling the peritoneal cavity to the bladder. Accordingly, ascites fluid from the peritoneal cavity will flow to the bladder due to a pressure gradient between the peritoneal cavity and the bladder. Furthermore, the electronic pump and manual pump may be arranged to create a bolus of fluid at varying time intervals in order to clear any blockages that may have formed within the shunt device. The manual pump may only be required to create a bolus of fluid when the electronic pump is unable to create a bolus of fluid due to a malfunction or loss of power. The electronic pump activates automatically based on a pre-programmed time interval to allow a subject to not have to manually pump the shunt device in order to clear any biological material form the shunt device.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A shunt for draining fluid, comprising:
a first catheter;
an electronic pump fluidly coupled to the first catheter;
a manual pump fluidly coupled to the electronic pump; and
a second catheter fluidly coupled to the manual pump,
wherein, during a drainage operation, the shunt arranged and configured such that a fluid is passively pressure-driven through the shunt, and the electronic pump is arranged to move a bolus of the fluid through the shunt.

2. The shunt of claim 1 further comprising a control unit that operates the electric pump at preselected time intervals.

3. The shunt of claim 2, wherein the electric pump moves the bolus of fluid as a pressure wave of the fluid flowing through the shunt at a greater pressure than the fluid being pressure-driven through the shunt.

4. The shunt of claim 3 further comprising a one-way valve fluidly coupled to the second catheter.

5. The shunt of claim 4, wherein the first catheter, the electronic pump, the manual pump, the second catheter, and the one-way valve are serially arranged to form the shunt.

6. The shunt of claim 5, wherein the first catheter is configured to be arranged within a peritoneal space of a subject in order to drain fluid from the peritoneal space.

7. The shunt of claim 6, wherein the second catheter is configured to be arranged within a bladder of the subject in order to fluidly couple the peritoneal space of the subject with the bladder of the subject.

8. The shunt of claim 1, wherein the one-way valve inhibits fluid within the bladder from flowing through the shunt.

9. The shunt of claim 1, wherein the manual pump is arranged and configured to move a bolus of the fluid through the shunt based on a manual activation.

10. A shunt for draining a fluid from a peritoneal cavity, comprising:
a first catheter configured to be arranged within the peritoneal cavity of a subject;
an electronic pump fluidly coupled to the first catheter;
a manual pump fluidly coupled to the first catheter;
a second catheter configured to be arranged within a bladder of a subject, the second catheter fluidly coupled to the electronic pump and the manual pump; and,
a one-way valve fluidly coupled to the second catheter to inhibit a back-flow of the fluid from the bladder through the second catheter,
wherein the electronic pump is arranged to create a bolus of the fluid in order to prevent blockages within the shunt, and the manual pump is arranged and configured to move a bolus of the fluid in order to inhibit blockages within the shunt when the electronic pump cannot create the bolus of fluid.

11. The shunt of claim 10 further comprising a control unit that operates the electronic pump at predetermined time intervals.

12. The shunt of claim 11, wherein the electronic pump moves the bolus of fluid as a pressure wave of the fluid flowing through the shunt at a greater pressure than the fluid being passively pressure-driven through the shunt.

13. The shunt of claim 12, wherein the electronic pump and the manual pump are configured to be arranged outside of the peritoneal cavity.

14. The shunt of claim 13, wherein the first catheter, the electronic pump, the manual pump, the second catheter, and the one-way valve are serially arranged to form the shunt.

15. The shunt of claim 14, wherein the electronic pump and the manual pump are fluidly coupled in parallel.

16. A method of draining a peritoneal cavity of a fluid, comprising:
arranging a first catheter within the peritoneal cavity of a subject;
arranging a second catheter within a bladder of the subject, wherein the first catheter and the second catheter are fluidly coupled;
passively flowing the fluid from the peritoneal cavity to the bladder through the first catheter and the second catheter due to a pressure differential between the peritoneal cavity and the bladder;
moving a first bolus of fluid via an electronic pump fluidly coupled to first catheter and the second catheter;
moving a second bolus of fluid via a manual pump fluidly coupled to the first catheter and the second catheter when the electronic pump cannot create the first bolus of fluid; and
flowing the first bolus of fluid or the second bolus of fluid through the second catheter in order to inhibit a blockage within the second catheter.

17. The method of claim 16, wherein moving the first bolus of fluid and the second bolus of fluid are moved at discrete time intervals.

18. The method of claim 17, wherein the first bolus of fluid and the second bolus of fluid are pressure waves of the fluid flowing through the second catheter at a greater pressure than the fluid flowing from the peritoneal cavity to the bladder through the second catheter due to the pressure differential between the peritoneal cavity and the bladder.

19. The method of claim 18, wherein a one-way valve is fluidly coupled to the second catheter in order to inhibit a back-flow of the fluid.

20. The method of claim 19, wherein the electronic pump and the manual pump are only used to move the first bolus of the fluid or the second bolus of the fluid.
